(19) United States
(12) Patent Application Publication
(10) Pub. No.: US 2018/0000685 A1
(43) Pub. Date: Jan. 4, 2018

(54) NEUROLOGIC THERAPY DEVICES, SYSTEMS, AND METHODS

(71) Applicant: NeuroRhythmics, Inc., Norwood, MA (US)

(72) Inventors: Carol M. MALONEY, Norwood, MA (US); Mark ETTLINGER, Lexington, MA (US)

(21) Appl. No.: 15/200,553

(22) Filed: Jul. 1, 2016

(51) Int. Cl. A61H 23/00; A61N 1/04; A61N 5/06; A61N 1/36; A61N 5/067

(52) U.S. Cl. CPC A61H 23/00; A61N 5/0622; A61N 1/36014; A61N 1/36025; A61N 5/0618; A61N 1/0456; A61N 2005/0645; A61N 2005/0659; A61N 2005/0662; A61N 2005/067

(57) ABSTRACT

A neurologic therapy device that is useful for rehabilitating patients suffering from one or more speech, and methods using the same. In embodiments the neurologic therapy device includes a band, one or more actuators on or within the band, and a control system. The control system is configured to cause the actuators to produce stimuli in accordance with a stimulus pattern. When applied to a body part (e.g. stimulus locations) of a user, the stimuli may assist the user to overcome or reduce one or more symptoms of a neurologic disorder. For example, the stimuli may assist an aphasia patient to initiate and/or cadence his/her speech.

### FIELD

The present disclosure generally relates to neurologic, cognitive and speech therapy devices and therapy methods using the same. More particularly, the present disclosure relates to therapy devices that are useful for various speech impediments and cognitive disorders, such as but not limited to aphasia, autism and symptoms of Parkinson's disease.

### BACKGROUND

According to the National Institute on Deafness and Other Communication Disorders (NIDCD), between 6-8 million individuals have some form of language disorder. Such language disorders may be brought on by various triggering events, such as a stroke, a brain injury, a neurological disorder, dementia, a brain tumor, or the like. Aphasia is one common language disorder that may result from such conditions.

Generally, aphasia is a language disorder that can affect many or all aspects of language, such as a patient's ability to speak, write, read, comprehend and understand words. In many cases aphasia results from damage to portions of a patient's brain that are responsible for language. For example, aphasia can arise from a cerebral vascular incident such as a stroke, though other causes such as a brain injury and cerebral trauma are also possible.

Many forms of aphasia exist, with some forms being more severe than others. Global aphasia is one severe form of aphasia, and may be characterized by a patient exhibiting abnormal speech initiation, word repetition, language comprehension, and word finding. In contrast, Broca's aphasia is a comparatively less severe form of aphasia that is characterized by a patient exhibiting abnormal speech initiation, abnormal word repetition, impaired word finding, and diminished memory and language comprehension. A patient suffering from Broca's aphasia may therefore be unable to speak long phrases and may have difficulty writing, but may be able to understand spoken or written words.

Given the significant role that written and verbal communication play in society, substantial effort has been dedicated to developing therapies to assist individuals suffering from aphasia to communicate effectively. One example of such therapy is “hand movement therapy,” which can assist a patient suffering from aphasia to initiate and/or cadence their speech through the use of hand motions. For example, patients employing hand movement therapy may be trained to draw a line on a piece of paper for each syllable to be spoken. Alternatively or additionally, the patient may move simply his/her hand for each syllable and/or word to be spoken.

Although hand movement therapy can improve the speech of patients suffering from various forms of aphasia, to be effective they generally require a patient to move his or her hand in some manner such as “tapping.” Such movement may be distracting to individuals with whom the patient is speaking, and may be considered by the patient to be embarrassing. In a public setting, for example, the movement of a patient's hands or fingers may be perceived as twitching and may provoke undesirable reactions from observers. Many aphasia patients therefore feel anxious when speaking in a public setting while using hand movement therapy. Moreover, hand movement therapy generally requires at least one of the patient's hands be occupied while communicating. Its use may therefore limit the ability of a patient to multitask while conversing. For example, tasks such as speaking on a phone while doing another activity (cooking, cleaning, writing, driving, etc.) may be quite difficult for a patient employing hand movement therapy.

### BRIEF DESCRIPTION OF THE DRAWINGS

Features and advantages of embodiments of the claimed subject matter will become apparent as the following Detailed Description proceeds, and upon reference to the Drawings, wherein like numerals depict like parts.

FIG. 1 is a perspective view of one example of a speech therapy device consistent with the present disclosure.

FIG. 2 depicts one example of a haptic actuator that may be used in accordance with the present disclosure.

FIG. 3 depicts a first example of a body facing surface consistent with the present disclosure.

FIG. 4 depicts a second example of a body facing surface consistent with the present disclosure.

FIG. 5 depicts a third example of a body facing surface consistent with the present disclosure.

FIG. 6 depicts one example of an electrical actuator consistent with the present disclosure.

FIG. 7 depicts one example of an optical actuator consistent with the present disclosure.

FIG. 8 is a block diagram of one example of a control system consistent with the present disclosure.

FIG. 9 depicts one example of a speech therapy device consistent with the present disclosure.

FIG. 10 depicts another example of a speech therapy device consistent with the present disclosure.

FIG. 11 is a flow chart of example operations of an example speech therapy method consistent with the present disclosure.

FIG. 12 depicts another example of a speech therapy device consistent with the present disclosure.

FIG. 13 depicts another example of a speech therapy device consistent with the present disclosure.

### DETAILED DESCRIPTION

In general, hand movement therapies leverage movement of the hand/finger(s) to help patients suffering from aphasia and other speech disorders to initiate and/or cadence their speech. Although they can be effective, the underlying reason for the effectiveness of hand movement therapy is still the subject of research. Without wishing to be bound by theory, the inventors believe that at least some of the effectiveness of hand movement therapy is attributable to the stimulation of one or more nerves in the wrist and/or arm, e.g., by the movement of a patient's hand. More specifically, the inventors believe that movement of a patient's hand or fingers in accordance with hand movement therapy causes the transmission of nerve signals through one or more nerves in the wrist that are connected to or associated with portions of the brain that are responsible for one or more aspects of language (e.g., speech initiation, cadence, etc.). Moreover, rhythmic, melodic, and/or intonation therapies are also believed to result in the reorganization of certain pathways within the brain, resulting in improved speech and cognition. In any case, such therapies can help a patient to cope with a language disorder by providing a means through tactile or sensory stimulus to cue the cognitive and, secondarily, the motor functions that enable speech and continued fluency.

Pursuant to that understanding the inventors developed the technologies described herein. As explained below, the technologies described herein leverage use of an artificial stimulus to one or more stimulus locations on a patient's body, such as on the hand, wrist, arm, or leg. The artificial stimuli may provide or cause the production of nerve signals in one or more nerves proximate the stimulus location(s). For example, in some embodiments the stimuli may provide or cause the production of nerve signals in the same or similar nerve pathways as melodic, rhythmic and hand movement therapy. In any case, the technologies described herein can produce therapeutic results that are similar to or even better than those achieved using hand movement therapy, without the need for a patient to move his/her body, and in particular his/her hand.

With the foregoing in mind the present disclosure generally relates to speech therapy devices and speech therapy methods using the same. In general the devices and methods described herein capitalize on the inventors' recognition that therapeutic benefits can be obtained by artificially stimulating one or more nerves in a patient suffering from one or more speech disorders such as aphasia. Indeed, in some instances, the devices and methods described herein may be as or more effective than traditional hand movement therapies for treating aphasia or another type of speech disorder/impediment, without requiring a patient to move his/her hand or another portion of their body.

It is noted that for the sake of clarity and ease of understanding, the technologies described herein are often described in the context of rehabilitating a patient suffering from aphasia. It should be understood, however, that the devices and methods described may be useful for rehabilitation of speech disorders other than aphasia, such as but not limited to stuttering, apraxia, other brain trauma (e.g. PTSD (Post Traumatic Stress Disorder)), cognitive functions resulting from autism and Parkinson's Disease, autism, pervasive development disorders, dementia, cerebral vascular disease and left lobe deficiencies, combinations thereof, and the like.

As used herein, the term “rehabilitation” means the improvement of a clinical outcome, for example, alleviation and/or reduction of the symptoms of a speech disorder such as aphasia, including improved speech initiation, word repetition, language comprehension, word finding, or a combination thereof.

As used herein the term “stimulation” refers to an action that produces or causes the production of nerve signals. “Stimulation” encompasses both “natural” stimulation, e.g., resulting from the movement of a patient's body, and “artificial” stimulation, e.g., resulting from the use of something external to the body, such as but not limited to an actuator.

One aspect of the present disclosure relates to speech therapy devices that are useful for treating one or more speech disorders, such as aphasia. In general, the speech therapy devices described herein are in the form of a wearable device that includes at least one actuator that is configured to provide an artificial stimulus (also referred to herein as a “stimulus” or “stimuli”) to a stimulus location on a patient's body. The stimulus may be configured to stimulate one or more nerves proximate a stimulus location. That is, the stimulus may provide or cause the production of nerve signals in one or more nerves proximate to a stimulus location.

The form of stimulation provided by the actuators may vary considerably. For example and as will be described below, the actuators may be configured to provide haptic stimulation (e.g., vibration, pressure, etc.), optical stimulation, electrical stimulation, another form of (artificial) stimulation, combinations thereof, and the like. The actuators may also be positioned and/or controlled to apply stimuli to specific stimulus locations, so as to stimulate specific nerves in a patient's body.

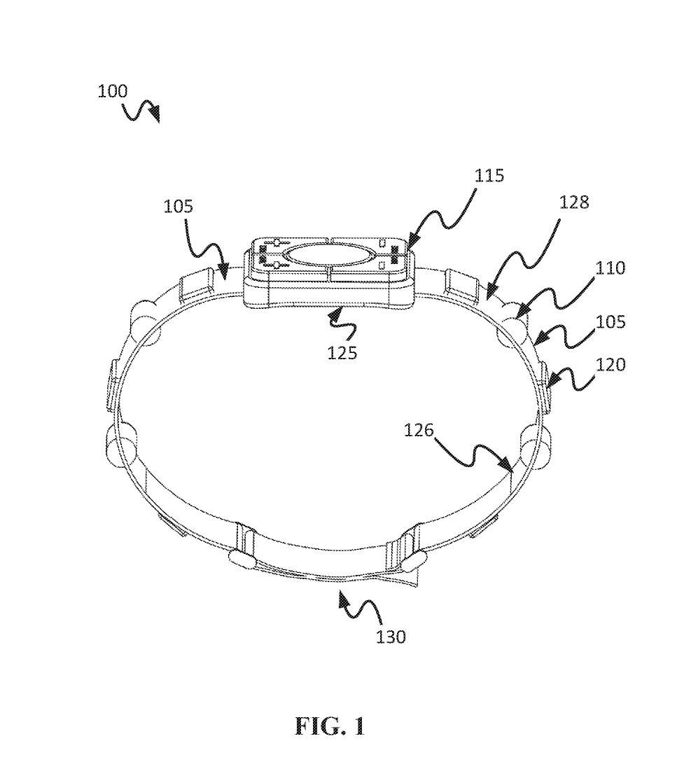

Reference is therefore made to FIG. 1, which illustrates one example speech therapy device consistent with the present disclosure. As shown, speech therapy device 100 includes a band 105, a plurality of actuators 110, a control system 115, and a power source 120. In some embodiments and as shown in the embodiment of FIG. 1, device 100 may include a housing 125. Housing 125 may be coupled to band 105 and may be coupled to or include control system 115. Device 100 further includes a power source 120, which may be located at any suitable location. For example and as shown in the embodiment of FIG. 1, power source 120 may be located in band 105. Alternatively or additionally, power source 120 may be located at another portion of device 100, such as within housing 125, control system 115, or the like.

Band 105 is generally configured to house or otherwise support other components of speech therapy device 100 and to provide a mechanism for speech therapy device 100 to be worn by or otherwise coupled to a patient. In that regard, device 100 in some embodiments may be in the form of a wearable device that may be worn on or about one or more body parts of a patient, such as but not limited to the patient's palm, wrist, lower arm, upper arm, chest, thigh, ankle or a combination thereof. Of course such locations are enumerated for the sake of example only, and it should be understood that device may be worn or coupled to any suitable body part of a patient. Without limitation, in some embodiments band 105 is configured such that device 100 may be worn about the wrist, arm or ankle of a patient.

As further shown in FIG. 1 one or a plurality of actuators 110 may be disposed on a (e.g., upper or lower) surface of band 105, within band 105, or some combination thereof. As will be explained in detail later, actuators 110 may independently or collectively function to provide a stimulus (e.g., haptic stimuli, electrical stimuli, optical stimuli, etc.) to one or more parts of a patient's body underlying device 100 (e.g., one or more stimulus locations). The applied stimulus may stimulate one or more nerves in a patient's body that are proximate to a stimulus location, causing the production of nerve signals which may help the patient address one or more symptoms of a speech disorder. For example the stimulus provided by actuators 105 may be applied in a stimulus pattern, wherein the stimulus pattern causes the production of nerve signals that can aid a patient to initiate speech, cadence speech, or to provide another therapeutic benefit to address one or more other symptoms arising from a speech disorder such as aphasia.

The fit of band 105 may impact the effectiveness of the stimulation provided by actuators 110. For example, if band 105 is ill fitting, actuators 110 may not be maintained in sufficiently close proximity to the body of the patient. The stimulus provided by actuators 110 may therefore be inadequately transferred to the patient's body, resulting in a less than desirable amount of nerve stimulation. This may be of particular concern in instances where one or more of actuators 110 is/are haptic actuators that are configured to provide a haptic stimulus. It may therefore be desirable to configure band 105 such that it may be adjusted to properly fit with a relevant portion of a patient's body. In some embodiments, such adjustment may cause band 105 to urge or more of actuators 110 in close proximity with a patient's body e.g., so that all or a portion of each actuator 110 is pressed against and/or protrudes into a patient's skin (preferably without causing discomfort).

Band 105 may be configured such that its size and fit may be adjusted. For example, in some embodiments band 105 may be one of a set of bands that may be interchangeably coupled to housing 125. In such instances each band in the set may include a first and second end that is configured to releasably engage with corresponding parts of housing 125 and/or control system 115. This concept is generally shown in FIG. 1, which depicts two parts of band 105 as being coupled to housing 125.

In instances where band 105 is one of a set of bands, each band in the set may differ in size and/or configuration relative to other bands in the set. For example, in some embodiments each band in the set may differ in size from other bands in the set, but otherwise may be substantially the same. Alternatively or additionally, the position of actuator(s) 110 and/or power source 120 may differ between the bands in the set. In that way, the fit of speech therapy device 100 and/or the position of actuators 110 may be adjusted by exchanging band 105 for another band in a set of bands.

In addition to providing a configurable fit (e.g., with a set of multiple bands that are identical except insofar as their size), the use of a set of interchangeable bands may also provide other customization opportunities. For example, in some embodiments device 100 may include a set of bands that includes at least a first band and a second band, wherein the first band is configured as a “training” or “therapy” band, whereas the second band is a “maintenance” band. The first (i.e., training) band may have a fit and actuator configuration that is designed to facilitate teaching an (untrained) user to use speech therapy device 100. In contrast, the second (i.e., maintenance) band may be configured for use by a trained patient, e.g., during conversations. As may be appreciated, the fit, locations of actuator(s) 110 and/or the power requirements between the first and second bands may differ. For example, relative to the second band the first band may be configured to provide a stronger (high intensity) stimulus, a stimulus that is applied to a larger area of the patient's body, combinations of different types of stimuli (e.g., haptic, optical, electrical, etc.), etc. combinations thereof, and the like. In contrast, relative to the first band the second band may have a fit and actuator configuration that is designed to provide a weaker (lower intensity) stimulus, to provide stimuli's that are relatively localized to specific parts of a patient's body, to provide a single type of stimuli or a combination of stimulus types, etc., combinations thereof, and the like. Alternatively or additionally, a clinician (e.g., a speech therapist) may utilize a set of bands (wherein each band differs from each other band in some manner) to test the effectiveness of different band configurations on the speech of a particular patient.

In any case, band 105 may be configured such that it may substantially conform to the relevant body part of the patient. In that regard, all or a portion of band 105 may be constructed from materials may elastically deform around and compress against a body part of a patient. For example, all or a portion of band 105 may be constructed from or include an elastomeric material, such as but not limited to a polysiloxane, latex, polyurethane, rubber (synthetic or natural), or some combination thereof. Alternatively or additionally, band 105 may include other materials, such as, but not limited to, metals (e.g., aluminum, stainless steel, or the like), either alone or in combination with elastomeric materials as noted above. In the latter case the inner surface of band 126 may have a curvature or other surface profile that may generally conform to a corresponding body part of a patient. In some instances, band 105 may be produced from a mold of a body part of a patient (e.g., a patient's wrist), such that the inward facing surface 126 thereof generally conforms to the patient's body.

In any case, band 105 may be configured such that it may be disposed around and a body part of the user. For example, in some embodiments, band 105 may be formed from or include one or more elastic materials (such as those noted above), and may (e.g., alone or in combination with control unit 115) form all or a part of continuous ring that may at least partially surround a patient's body part, such but not limited to the patient's hand, wrist, forearm, upper arm, chest, thigh, calf, ankle, or foot. To facilitate sizing or for another purpose, band 105 may include one or a plurality of links that may be removed or added to adjust the size of band 105 to a particular patient. Similarly, in some embodiments band 105 may also include a clasp, which may be moveable to adjust the diameter of device 100 or, more particularly, of band 105. That concept is shown in FIG. 1, which depicts device 100 as including a band 105 having first and second parts (not labeled) that are each coupled to control system 115/housing 125, and which are coupled to one another by clasp 130.

As will be described in further detail later, actuators 110 are generally configured to apply or cause the application of a stimulus to a portion of a patient's body, i.e., at one or more stimulus locations. The application of the stimulus to one or more stimulus locations may stimulate nerves proximate such locations, resulting in the production of nerve signals which may help the patient to address one or more complications arising from a speech disorder. In that regard, actuators 110 may be configured to provide a haptic, optical, or electrical stimulus, or one or more combinations thereof.

While it may be desirable in some instances to configure device 100 such that a stimulus is localized to stimulus locations proximate to an actuator 110 providing the stimulus, in some instances another configuration may be desired. For example, it may be desirable to configure device 100 such that a stimulus is applied to a stimulus location that is remote from an actuator 110 giving rise to the stimulus. That is, it may be desirable to configure device 100 such that a stimulus generated by one or more actuators 110 is transferred to (e.g., detectable by a patient at) various stimulus locations on the patient's body, including one or more stimulus locations that is/are remote from the actuator 110 that produces the applied stimulus.

Device 100 may therefore be configured such that a stimulus provided by an actuator 110 located at a first location on/within band 105 is at least partially applied to a stimulus location on the patient's body that is remote from the first location. To accomplish that, band 105 and/or actuators 110 may be configured such that a stimulus provided by an actuator(s) 110 is transmitted at least partially through band 105. In other words, band 105 may be configured such that a stimulus generated by an actuator 110 at a first location of band 105 may be distributed through all or some larger portion of band 105. In such instances generation of the stimulus at the first portion of band 105 (e.g., by one or more actuators 110) may be felt or otherwise detected by a user over a portion of their body that is larger than the relevant actuator(s) 110 and/or which is remote from the first location. For example, band 105 may be configured such that a user may perceive a vibratory stimulus provided by one actuator 110 as the vibration of one portion, multiple portions, or all of band 105.

In instances where transmission of a stimulus produced by actuators 110 through all or a portion of band 105 is desired, at least a portion of band 105 may be made of or include materials that facilitate transmission of a stimulus provided by actuator(s) 110 to other parts of band 105. For example where an actuator 110 is a haptic actuator configured to provide a haptic stimulus (e.g., a vibration, oscillation, or the like), all or a portion of band 105 may be formed from or include relatively rigid materials (e.g., metals, alloys, composites, etc.) that will conduct the haptic stimulus to other parts of band 105 that are remote from the actuator. For example, all or a portion of band may be formed from or include materials that vibrate in response to the haptic stimulus provided by the relevant actuator 110. Alternatively or additionally, all or a portion of band 105 may formed from or include an elastomeric body (or “matrix”) that includes elements (e.g., rods, discs, etc.) formed from materials that are relatively rigid (e.g., metals, alloys, etc.). In either case, a haptic stimulus provided by at least one actuator 110 at a first portion of band 105 may cause a corresponding stimulus (e.g., vibration, oscillation, or the like) at another (second) part of band 105. As may be appreciated, while such a configuration may limit the ability of device 100 to produce localized stimuli to a user, it may yield power savings by reducing the number of actuators 110 needed to provide a desired stimulus.

Alternatively or additionally, all or a portion of band 105 and/or actuator(s) 110 may be configured to provide localized stimuli to a part of a user's body. That is, band 105 and/or actuators 110 may be configured such that a stimulus provided by actuators 110 is localized (e.g., focused) to a stimulus location on the patient's body that is proximate to the actuator(s) producing the stimulus. In such instances, it may be desirable to limit the conduction/transfer of stimuli generated by actuator(s) 110 to the regions of band 105 that are proximate to such actuator(s) 110.

To facilitate stimulus localization, in some embodiments one or more portions of band 105 may include materials that do not react to or conduct a stimulus provided by one or more of actuators 110. For example when one or more of actuators 110 is a haptic actuator, at least some portion of band 105 may include damping (vibration damping or otherwise) material which may limit or even prevent the transmission of a haptic stimulus provided by the actuator to other portions of band 105. Non-limiting examples of damping materials that may be used include elastomeric materials, such as but not limited to elastomeric silicones, fluoroelastomers, natural or synthetic rubber (e.g., polyisoprene, polybutadiene, chloroprene, butyl rubber, nitrile rubber, etc.), ethylene vinyl acetate (EVA), thermoplastic polyurethanes, themolastic olefins, combination thereof, and the like. Similarly when one or more actuators 110 is an electrical actuator, it may be at least partially surrounded with one or more insulating (i.e., non-electrically conducting) materials. Still further, when one or more actuators 110 is an optical actuator, it may be at least partially surrounded with one or more materials that are substantially opaque to (i.e., which do not or substantially do not transmit) light emitted by one or more light sources of the optical actuator.

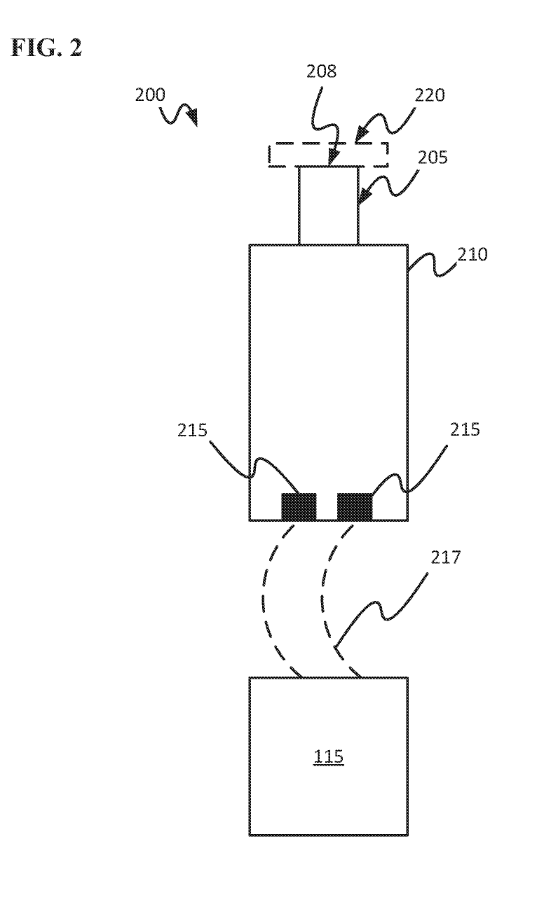

In instances where one of more actuators 110 is a haptic actuator, for example, stimulus localization may be accomplished by providing a damping material around all or a portion of a haptic actuator that is embedded into or otherwise coupled to band 105. More specifically and as shown in the embodiment of FIG. 1, band 105 may include an inward facing surface 126 (facing the patient), a body portion, and an outward facing surface 128. In such instances and as will be later described in conjunction with FIG. 2, one or more haptic actuators that include a drive and a body facing surface may be used as one or more of actuators 110. In any case the haptic actuator may be embedded into or otherwise coupled to band 105 such that its body facing surface is oriented towards inward facing surface 128, i.e., such that it faces the patient when device 100 is worn. Damping material may be disposed on or around at least a portion of the haptic actuator, so as to limit or even prevent the conduction of haptic stimuli through band 105 when the haptic actuator provides a haptic stimulus.

For example, in some embodiments damping material may be disposed around portions of actuator 110 other than its body facing surface (e.g., a drive portion thereof). That is, in some embodiments the sides and distal portion of the drive of an actuator may be surrounded with damping material, whereas the body facing surface of the actuator may be left exposed along inward facing surface 126. Alternatively or additionally, the body facing surface of the actuator(s) 110 may be covered with a material forming all or a portion of inward facing surface 126, wherein such material is configure to facilitate transfer of a stimulus to a stimulus location proximate to (e.g., underlying) the relevant actuator 110. In either case, the haptic actuator may provide a haptic stimulus which may be conveyed to the patient through the body facing surface thereof, but which may otherwise be absorbed or dissipated by the damping material. In that way, haptic stimuli may be limited to stimulus regions that are about the same size or smaller than the body facing surface of a haptic actuator. Similar techniques may be used to localize optical and electrical stimuli, with electrically insulating or optically opaque/reflective material being substituted for the damping materials used with haptic actuators.

As noted above, band 105 may be configured such that actuator 110 is fully or partially embedded therein. That concept is shown in FIG. 1, which depicts actuators 110 as being embedded fully or partly within band 105. It may be appreciated that in such embodiments, band 105 may be of a single or multilayer construction, wherein the materials of band 105 may envelope all or a portion of actuator 110. Moreover in such instances the location of actuators 110 within band may be relatively fixed, although the placement of actuators 110 relative to the patient's body may be adjusted by adjusting the size of band 105 and/or its rotational orientation (i.e., by rotating device 100 about the patient's body).

Although the configuration shown in FIG. 1 is useful, it may be desirable in some instances to enable a patient or other user (e.g., a speech therapist) to reposition the location of one or more actuators 110 relative to band 110. Therefore in some embodiments, device 100 is configured such that actuators 110 may be repositioned relative to band 105. In such embodiments the inward and/or outward facing surfaces 126, 128 of band 105 may include a plurality of receiving regions, wherein each receiving region is configured to couple to an actuator 110 and/or an engagement region thereof.

For example, the inward or outward facing surfaces 126, 128 of band 105 may include a plurality of receiving regions in the form of recesses, wherein each recess is configured to receive and mechanically engage (e.g., compress) with all or a portion of one or more actuators 110. In some embodiments, for example, the recesses within the inward/outward facing surface of band 105 and the actuators 110 may be complimentary in shape, such that one or more sides of the recesses compressively engage with corresponding portions of an actuator inserted into the recess. Alternatively or additionally, the recesses may be configured to mechanically engage with one or more portions of an actuator inserted therein. For example, the recesses may include a first component (e.g., a ridge or channel) that interlocks with a corresponding second component (channel or ridge) of the inserted actuator. In any case, all or a portion of the actuator 110 may extend from or be substantially coplanar with proximate portions of the inward or outward facing surfaces of the band 105.

Alternatively or additionally, in some embodiments the receiving regions of band 105 are configured to couple with engagement regions of an actuator 110, such that all or substantially all of the actuator 110 is disposed on the inward or outward facing surfaces 126, 128 of band 105. For example, in some embodiments band 105 may include a plurality of receiving regions that are defined by one or more ridges or grooves, which are configured to mechanically couple with corresponding engagement regions (e.g., complimentary shaped grooves or ridges) of actuator 110. In such instances, the actuators may be slidably coupled to band 105, such that they may be slid around at least a portion of the inner or outer circumference thereof to a desired position. Alternatively or additionally, the receiving regions and engagement regions may be configured as opposing parts of a snap fastener, thereby enabling a patient or other user to couple one or more actuators to band 105 via a snap.

Still further, in some embodiments band 105 may include a plurality of receiving regions in the form of pockets that are sufficiently sized to allow for the insertion of one or more actuators 110 therein. The pockets may include at least one opening, e.g., along one or more sides of the body portion of band 105. The opening(s) may include or be in the form of one or more pockets that extend at least partially through a body portion of band 105, e.g., in a direction substantially perpendicular (i.e., normal) to the inward and outward facing surfaces thereof. In some embodiments the pockets are at least partially defined by a fabric or other material, which may be the same or different from the materials used to form other portions of the band. Furthermore, each pocket may have at least one open side that can be closed by a variety of mechanisms to facilitate retention of an actuator therein. For example, the open side(s) of each pocket may include a hook and loop fastener, buttons, snaps, magnets, or other closing means.

As may be appreciated, configuring device 100 such that actuators 110 may be repositioned relative to band 105 may provide one or more advantages to a patient or another user such as a speech therapist. For example, their use may allow a patient or other user to dynamically configure device 100 (e.g., by repositioning one or more actuators 110) to target the application of a stimulus to particular stimulus locations on a patient's body (and hence, one or more nerves proximate such locations). This may be useful in instances where the therapeutic effect provided by device 100 may be diminished due to nerve fatigue or another reason. Indeed in such instances a patient or other user may reposition actuators 110 relative to band 105 so as to target other stimulus locations/nerve(s), potentially allowing the previously stimulated locations/nerve(s) to recuperate. Similarly, the ability to reposition actuators 110 along band 105 may allow a patient or speech therapist to account for natural variation in the location of nerves between patients.

As will be described in detail later, actuators 110 are generally configured to produce a stimulus in response to control signals issued by control system 115, e.g., in accordance with a desired stimulus pattern. That being said, for the sake of clarity the present disclosure will now proceed to describe the nature and function of actuators 110. Following such discussion, the interaction of actuators 110 and control system 115 will be explained.

With the foregoing in mind, as noted above device 100 may include one or a plurality of actuators 110, wherein each actuator may be configured to provide the same or a different type of stimulus. The type of stimulus provided by actuator(s) 110 may vary widely and, in some instances, a single actuator may provide one or multiple types of stimuli. For example, in some embodiments the actuators described herein may be configured to provide haptic stimuli, electrical stimuli, optical stimuli, combinations thereof, and the like.

The type and number of actuators 110 included in device 100 is not limited. Therefore while FIG. 1 depicts an embodiment in which four actuators 110 are used, it should be understood that such illustration is for the sake of example only and that any suitable number of actuators may be used. For example, device 100 may include greater than or equal to about 1, 2, 5, 10, 15, 25, 50, 100, or even 1000 actuators or more, wherein the position or some or all of the actuators 110 is fixed or moveable relative to band 105.

In various embodiments device 100 may include relatively few (e.g., about 1, 2, 3, 4 or 5) actuators, wherein the position of each actuator 110 relative to band 105 is fixed or repositionable, as discussed above. In such instances it may be understood that the number of actuators 110 may be insufficient to directly stimulate all portions of the patient's body underlying the inner surface of band 105. Alternatively, in other embodiments device 100 may include a relatively large number of actuators, e.g., greater than 5, 10, 15, 20, 50, 100, 1000, etc.), wherein the position of each actuator 110 relative to band 105 is fixed or repositionable, as discussed above. For example, in such embodiments the number of actuators may be sufficient to enable stimulation of all or substantially all of the patient's body underlying the inner surface of band 105. In either case, control system 115 may control the actuators 110, such that a desired stimulus pattern is provided to the patient.

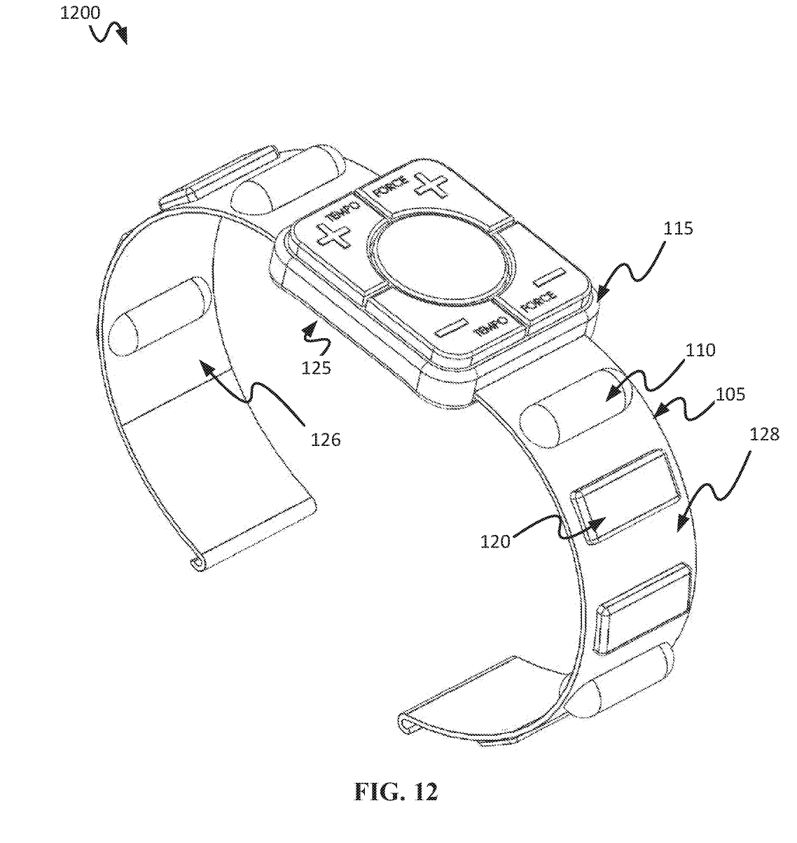

In embodiments in which device 100 includes a plurality of actuators 110, their number and placement may be diversified. For example, a plurality of actuators 110 may be positioned along band 105 such that they are proximate to specific nerves or nerve bundles of the patient when device 100 is worn. The proximity of actuators 110 to the nerves may be adjusted, for example, by adjusting the fit (e.g., tightness) of band 105, by repositioning actuators 110 along band 105, or a combination thereof. Even in instances where actuators 110 are not repositionable, the devices of the present disclosure are not limited to the configuration shown in FIG. 1. For example, in some embodiments the fixed position of actuators 110 relative to power source 120 may be different than the configuration shown in FIG. 1. This concept is illustrated in FIG. 12, which depicts one example of a device 1200 that is consistent with the present disclosure, wherein the positions of actuators 110 and power source 120 have been flipped. While FIG. 12 illustrates such a configuration without clasp 130 or other closing means, it should be understood that clasp 130 or other closing means may be used.

In instances where device 100 is configured to be worn about the wrist or arm of a patient, for example, one or more actuators may be positioned along band 105 such that it/they are proximate one or more of the radial, ulnar, and/or media nerves of the patient. For example, in some embodiments at least one actuator may be disposed proximate the radial nerve, the ulnar nerve, or the media nerve. In other embodiments, device 100 includes at least first and second actuators, wherein the first and second actuators are configured and positioned so as to stimulate a first nerve and a second nerve, respectively, wherein the first and second nerves are different from one another and are chosen from the radial, media, or ulnar nerves of a patient. In any case where multiple actuators are used, the actuators may be configured to provide the same or different times of stimulation.

More specifically, in some instances device 100 may be configured to be worn about the wrist of a patient. In such instances and as shown in FIG. 1, actuators 110 in some embodiments may be positioned at various locations on or within band 105. For example in the illustrated embodiment, first, second, third, and fourth actuators 110 may be present at first, second, third, and fourth locations along band 105. The first, second, third, and fourth locations may be selected such that when device 100 is worn on a patient's wrist, the first actuator 110 is be positioned on the volar-side (palm side) of the wrist, the second actuator 110 is positioned on the dorsal-side (back side) of the wrist, and the third and fourth actuators are positioned on the lateral sides of the wrist. In such instances, the first through fourth actuators may be configured to provide the same or different types of stimulation.

In some embodiments a different actuator type may correspond to each nerve and/or body part region being stimulated. For example, when speech therapy device 100 is worn on the wrist of the user, an actuator 110 may correspond to each of the radial nerve, ulnar nerve, and median nerve. In these instances, a different actuator type may correspond to each nerve. Alternatively, in some embodiments, each nerve may have a corresponding actuator 110 of the same type. In some instances, diversifying actuator types may reduce nerve fatigue and/or allow for the generation of more complex stimulus patterns. For example, when using only one actuator type there may be cross-stimulation between nerves and/or the sensation felt at the nerves may be dulled due to the repetitive stimulation using a single actuator type.

In some instances it may not be desirable to place a specific emphasis on positioning an actuator in close proximity (e.g., directly over) a nerve to be stimulated. Instead, it may be desirable to generally stimulate nerves in a portion of the patient's body that are remote from the position of a particular actuator generating the stimulus. To that end, in some instances actuator 110 may be configured and positioned so as to provide a stimulus as described above, wherein the stimulus is transmitted to one or more nerves in some manner, such as via bone conduction, skin conduction, or the like. In the case of bone conduction, a stimulus provided by one or more actuators 110 may travel from the actuator through one or more bones that are proximate one or more nerves to be stimulated. Stimulation of a desired nerve may then result via the conduction of the stimulus through one or more bones. For example, when speech therapy device 100 is worn on a wrist of the user, actuator 110 may be configured to transmit, via one or more bones of the arm, a stimulus (e.g., a haptic stimulus such as a vibration) to one or more of the radial nerve, ulnar nerve, median nerve, or any other nerve that is located remotely from the position of actuator 105.

The type and design of actuator 110 may also have an effect on the performance of speech therapy device 100. For example, the physical design of actuator 110 may change how the stimulus is transmitted to the user, the type of stimulus that is provided, and various other characteristics. In that regard and as noted above, actuators 110 may be in the form of or include haptic actuators, electrical actuators, or optical actuators. In some embodiments all or a portion of actuators 110 are haptic actuators that are configured to provide haptic stimulation to one or more stimulus locations on a patient's body, either alone or in combination with another form of stimulation. For example, in some embodiments actuators 110 include at least one first actuator 110 configured to provide haptic stimulation, and at least one second actuator configured to provide another form of nerve stimulation, such as electrical or optical stimulation. In additional embodiments, all of actuators 110 are configured to provide electrical stimulation, optical stimulation, or a combination thereof. In any case and as will be described below, the intensity, frequency, tempo, rhythmic patterns and other characteristics of the stimulation provided by the actuators described herein may be varied, e.g., in accordance with a fixed or variable stimulus pattern.

In general, haptic stimulation may be understood to be stimulation that may be detected by a patient's sense of touch. Non-limiting examples of haptic stimulation include vibration, oscillation, pressure, combinations thereof, and the like. Without limitation, in some embodiments one or more (e.g., all) of actuators 110 are configured to provide haptic stimulation in the form of vibration, pressure, or a combination thereof. In any case and as will be described below, the intensity, frequency, and other characteristics of the haptic stimulation provided by such actuators may be varied, e.g., in accordance with a fixed or variable stimulus pattern.

In instances where one or more of actuators 110 is a haptic actuator, any suitable haptic actuator may be used. Non-limiting examples of suitable haptic actuators that may be used for that purpose include eccentric mass motor actuators, rolling actuators, voice coil actuators, solenoid actuators, piezo-electric haptic actuators, magnetic/electromagnetic haptic actuators (e.g., a rotary magnet with a ferromagnetic fluid bladder, linear actuators a rotary magnet with a ferromagnetic actuator, and a solenoid electromagnet with a ferromagnetic bladder), combinations thereof, and the like. Without limitation, in some embodiments the speech therapy devices employ one or a plurality of eccentric mass motor actuators.

Although the type of haptic actuators that may be used in accordance with the present disclosure is not particularly limited, the configuration of such actuators may have an impact on the performance of device 100. It may therefore be desirable in some embodiments to utilize one or more haptic actuators that are configured in a particular manner, e.g., so as to facilitate the application of a desired haptic stimulus to a stimulus location on a patient's body.

Reference is therefore made to FIG. 2, which depicts one example of a haptic actuator 200 that may be used in a speech therapy device consistent with the present disclosure. Generally, haptic actuator 200 is configured to impart a haptic stimulus to a wearer of device 100. In that regard, haptic actuator 200 includes a transducer 205, a driver 210, and (optional) contacts 215. When used, optional contacts may couple haptic actuator 200 to control system 115, e.g., via one or more control lines. It should be understood that wired communication between haptic actuator 200 (or any other actuator described herein) is not necessary, and that haptic actuator (or any other suitable actuator) may be configured to receive control signals from control system 115 wirelessly. For example, the control systems described herein may establish a wireless communication link with the actuators described herein using any suitable wireless communication protocol (802.11b, 3G, 4G, long term evolution (LTE), ZigBee, near filed communication (NFC), Wi-Fi, etc.). In either case, haptic actuator 200 may be configured to receive control signals 217 from control system 115.

In response to control signals 217, haptic actuator 200 may produce a haptic stimulus that may be at least partially conveyed by transducer 205 to a stimulus location on the body of a patient wearing device 100. For example in response to control signals 217, driver 210 or a component thereof may cause transducer 205 to vibrate, oscillate, or the like, e.g., in accordance with a desired stimulus pattern. Due to the positioning of actuator 200, the intensity of the movement of transducer 205, and/or other factors (e.g., the materials of band 105 and/or any covering thereover, the proximity of actuator 200 to the body of the patient, etc.) the haptic stimulus may be conveyed to one or more stimulus locations on the body of a patient, resulting in the stimulation of nerves proximate such locations.

In some instances transducer 205 may also be configured such that is moveable between a retracted position and an extended position, e.g., in response to a control signal 217. Such movement may be independent of or simultaneous with the oscillation and/or vibration of transducer 205 noted above. In any case driver 210 may, in response to a control signal 217, cause transducer 205 to extend from a retracted position to an extended position, so as to impart pressure against a portion of the skin of a patient wearing device 100. Similarly, such pressure may be relieved by the retraction of transducer 205 by driver 210, e.g., in response to one or more control signals 217.

To facilitate transfer of a haptic stimulus to a patient's body and/or the stimulation of desired nerve(s), body facing surface 205 may have a surface 208 that is configured to conform to all or a portion of the patient's body, e.g., at a stimulus location. For example where device 100 is to be worn about a patient's wrist, surface 208 of haptic actuator 200 may be configured to conform to a stimulus location located on a relevant portion of the user's wrist such as the volar, dorsal, and/or lateral sides thereof. In that regard, in some instances surface 208 of body facing surface 405 may have a contoured shape that is complimentary with a corresponding portion of the shape of the patient's wrist.

For example, surface 208 may be formed from or include a flexible material which may conform to the surface of part of the patient's body when device 100 is worn. For example, all or a portion of surface 208 may be formed from an elastomeric material such as a siloxane, latex, polyurethane, rubber, or other material. Alternatively or additionally, the shape of the surface 208 of transducer 205 may be at least partly defined by relatively rigid materials, in which case the shape thereof may be defined based on an average body part (e.g., wrist) or a mold or other representation of the body part (e.g., wrist) of a particular patient. Similarly, in some embodiments surface 205 may be complimentary in shape to the inward facing surface 126 of band 105. In such instances, actuator 200 may be disposed on or within band 105 such that surface 208 thereof is coplanar or substantially coplanar with adjacent portions of inward facing surface 208.

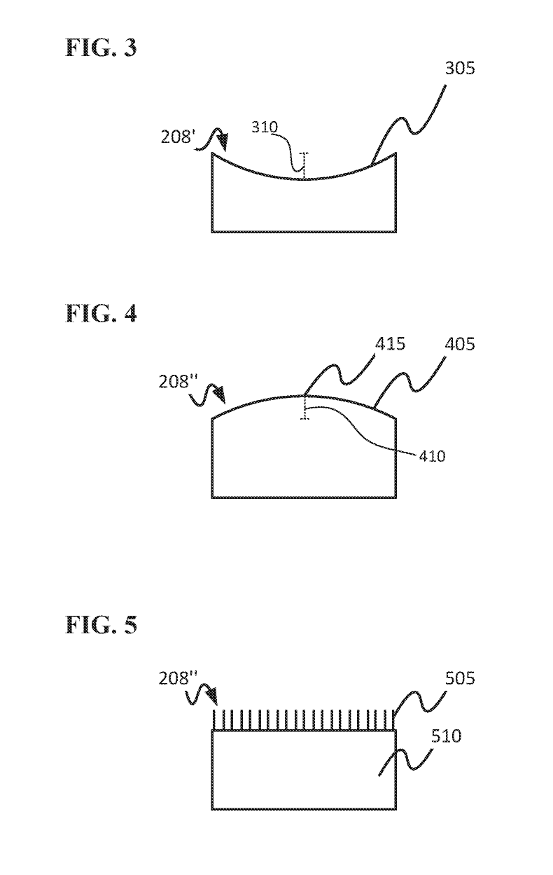

Reference is now made to FIG. 3, which depicts one example of a body facing surface that may be used in a haptic actuator 200 consistent with the present disclosure. As shown, surface 208′ has an arcuate shape that is configured to generally conform to all or a portion of a stimulus location on a patient's body, such as the locations noted above. In this embodiment, surface 208′ includes an arcuate surface 305 with a radius 310 that is sized to receive a portion of the patient's body. For example, radius 310 may be defined such that surface 208′ may generally conform to all or a portion of the curvature of the volar, lateral, or dorsal sides of a patient's wrist. In such instances at least a portion of patient's wrist may rest on and/or within arcuate surface 305 when device 100 is worn.

FIG. 4 depicts another example of a body facing surface that may be used in a haptic actuator consistent with the present disclosure. As shown, surface 208′ includes a bulge 405 having a radius 410 and an apex 415. Radius 410 may be generally configured such that when surface 208″ is pressed against a patient's skin, the distance between apex 415 and a nerve in the patient's body (e.g., wrist) may be reduced or even minimized (preferably without causing discomfort to the patient).

FIG. 5 depicts yet another example of a body facing surface that may be used in a haptic actuator consistent with the present disclosure. As shown, surface 208′″ includes a plurality of protuberances 505 extending from an underlying portion 510 of transducer 205. Protuberances 505 may be formed of any suitable material, and may be rigid, semi rigid, or flexible. As may be appreciated, the rigidity of 505 may facilitate the transmission of a stimulus generated by haptic actuator 200 to a body part of a patient. In contrast, the flexibility of protuberances 505 may make device 100 more comfortable to wear by a patient. It may therefore be desirable in some instances to select and/or control the stiffness of protuberances 505 to achieve a desired balance between patient comfort and sufficient transfer of stimuli produced by actuator 200 to a patient.

It is noted that FIGS. 3 and 4 depict examples of surface 208′, 208″ that include a single arcuate shape 305 or bulge 405. It should be understood that such illustrations are for the sake of example only, and that body facing surfaces 208′, 208″ may include any suitable number of arcuate shapes and/or bulges. For example, surface 208′ may include a plurality (e.g., 2, 3, 4, 5 or more) of arcuate shapes 305. Likewise, surface 208″ may include a plurality (e.g., 2, 3, 4, more, etc.) of bulges 405. Alternatively or additionally, transducer 205 of FIG. 2 may include a body facing surface that includes a combination of arcuate shapes and bulges.

Moreover, while FIGS. 3 and 4 depict body facing surfaces that do not include protuberances, it should be understood that protuberances may be included in such structures. For example, protuberances that are the same or similar to those shown in FIG. 5 may be provided on one or more parts of the arcuate shape(s) 305 and/or bulge(s) 405 of body facing surfaces 208′, 208″.

While the haptic actuators including a surface 208 configured in the manner described above are useful, other haptic actuator configurations may also be desirable. For example, it may be desirable to configure haptic actuators 200 with one or more interchangeable parts, e.g., which may enable a patient or other user to customize the fit and/or degree to which a stimulus is provided to a stimulus location. In that regard, in some embodiments the haptic actuators described herein may include a capping member coupled to a portion of a transducer. That concept is shown in FIG. 2, which illustrates the use of an optional capping member 220 with surface 208. As shown in the illustrated embodiment, optional capping member 220 may be coupled to surface 208 of transducer 205. It should be understood however that optional capping member need not be configured in that manner, and may be coupled to transducer 205 in any suitable manner.

Without limitation, in some embodiments optional capping member 208 may be releasable coupled to transducer 205 or another part of haptic actuator 200. In such instances a plurality of different optional capping members 220 may be interchangeably coupled to transducer 205 or another portion of haptic actuator 200. As may be appreciated, the interchangeability of optional capping member(s) 220 may allow a patient, speech therapist, or other user to customize the fit of device 100, to enhance or dull the transfer of haptic stimulation produced by haptic actuator 200 to a patient, or some combination thereof.

In general, optional capping member 220 may perform the same or similar function as body facing surfaces 208′-208′″ noted above. That is, optional capping member 220 may be configured to conform to all or a portion of a body part of a patient wearing device 100, such as but not limited to a stimulus location. For example when device 100 is to be worn on a patient's wrist, optional capping member 220 may be configured to conform to all or a portion of the patient's wrist.

Alternatively or additionally, optional capping member 220 may be configured to localize application of a haptic stimulus to a relatively small stimulus location, or to distribute application of a haptic stimulus to a relatively large stimulus location. In the former case, optional capping member 220 may have a tapered, point-like, bullet-like, or other shape, with a body contact surface that is relatively small as compared to the surface 208 of transducer 205. In such instances, only the relatively small body contact surface of optional capping member 220 may come into contact or close proximity to a patient's body. Application of the haptic stimulus may therefore be limited to a stimulus location that is about the size of the body contact surface of optional capping member 220, i.e., to an area smaller than that of surface 208. To the contrary, in the latter case optional capping member may have a body contact surface that has an area that is larger than that of surface 208. In such instances, application of a haptic stimulus may be distributed to a stimulus location that is about the size of the relatively large body contact surface of optional capping member 220, i.e., to an area that is larger than the area of surface 208.

Optional capping member 220 may be formed from any suitable material or combination of materials, such as but not limited to the elastomeric and relatively rigid material noted above as being suitable for forming surface 208. Moreover, optional capping member 220 may include a body contact surface that is configured in the same or substantially the same way as body facing surfaces 208′-208″.

As noted previously one or more haptic actuators 200 may be disposed on or within band 105 of device 100. For example, one or more haptic actuators 200 may be embedded within band 105 such that surface 208 of haptic actuator 200 is coplanar or substantially coplanar with a portion of inward facing surface 128 of band 105. In such instances, device 100 may be configured such that surface 208 of haptic actuator remains exposed, e.g., such that it may contact a patient's skin when device 100 is worn. In embodiments where a haptic actuator including an optional capping member is used, for example, it may be disposed on or within band 105 such that all or a portion of optional capping member 220 is coplanar or substantially coplanar with inward facing surface 126. Alternatively or additionally, one or more haptic actuators 220 may be disposed on or within band 105, such that surface 208 and/or optional capping member 220 is covered by the material(s) forming inward facing surface 126 of band 105.

In some embodiments one or more of actuators 110 may be or include electrical actuators that are configured to provide an electrical stimulus to a stimulus location on a body of a patient. In general, an electrical stimulus may be understood to be an electrical signal (e.g., an electric current) that may be applied to a stimulus location on a patient's body. The intensity, duration, and other characteristics of the applied electrical stimulus may be controlled by control system 115, and may be defined in accordance with a predefined or variable stimulus pattern, as will be discussed later.

The type and nature of electrical actuators that may be used is not particularly limited, and any suitable electrical actuator may be used as or in one or more of actuators 110. In some embodiments the electrical actuators described herein may include or be in the form of or more electrodes (e.g., electromyographic electrodes, peripheral nerve stimulation electrodes, electroencephalogram electrodes, electrocardiogram electrodes, combinations thereof, and the like) that are configured to contact a stimulus location on a patient's body, e.g., via direct contact with the patient's skin or through another mechanism. In some embodiments, nerve stimulation may be provided by such electrodes, e.g., in the same manner as performed in an electromyography and/or Transcutaneous Electrical Nerve Stimulation.

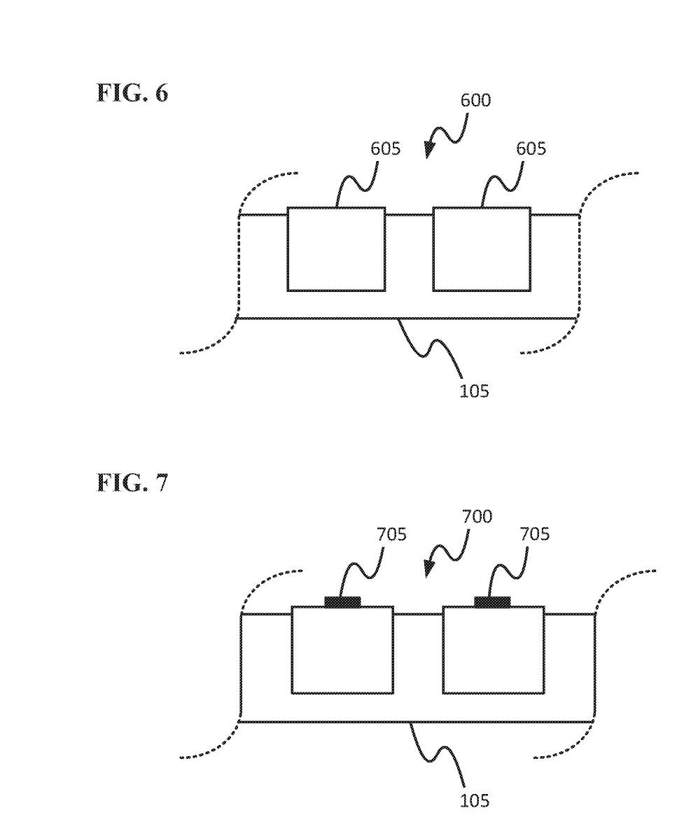

Reference is now made to FIG. 6, which depicts one example of an electrical actuator that may be used as or within one or more of actuators 110. As shown, electrical actuator 600 includes a plurality of electrodes 605, which may be embedded into or otherwise coupled to band 105. In some embodiments electrodes 605 are embedded within or otherwise coupled to inward facing surface 126 of band 105. In such instances at least a portion of electrodes 605 may remain exposed on the inward facing surface 126 of band 105, such that they may come into contact with a portion of a patient's skin when device 100 is worn.

In some embodiments a plurality of electrodes 605 are used in device 100, and are designed such that they come into contact with a patient's skin when device 100 is worn. In such instances the plurality of electrodes 605 may include at least a first electrode (e.g., an anode) and a second electrode (e.g., a cathode), wherein the first electrode is a positive terminal and the second electrode is a negative terminal. During the application of an electrical stimulus, a current may flow between the positive and negative terminal, e.g., via the patient's body. The voltage, amperage, duration, and other characteristics may be set by controller 115, e.g., in accordance with a fixed or variable stimulus pattern. Alternatively, in instances where a plurality of electrical actuators are used, a first electrical actuator may be configured as a positive terminal and a second electrical actuator may be configured as a negative terminal, with similar results.

In various embodiments the applied electrical stimulus may be configured to cause the production of nerve signals in one or more nerves disposed in stimulus location that is situated generally between one or more positive and negative terminals (e.g., between and first and second electrode as described above). It may therefore be desirable to configure the shape and placement of electrodes 605 so as to define a desired stimulus location on the patient's body. For example, increasing or decreasing the spacing of electrodes 605 may increase or decrease, respectively, the size of the stimulus location on the patient's body. In instances where repositionable first (e.g., positive) and second (e.g., negative) actuators are used, the size of the stimulus location may be defined by repositioning the first and second actuators relative to one another on band 105.

In some embodiments, one or more of actuators 110 may include or be in the form of an optical actuator. In general, an optical actuator may be understood as an actuator that can provide an optical stimulus to a stimulus location on the body of a patient wearing device 100. The optical stimulus may be light in one or more regions of the electromagnetic spectrum, such as the ultraviolet, visible, or infrared regions. For example, in some embodiments the optical stimulus may be a specular or diffuse light in the visible or infrared regions. In various embodiments, the optical stimulus may be one or more light impulses generated by a visible or infrared laser. In any case, the characteristics of the optical stimulus (e.g., its optical power, duration, etc.) may be controlled by controller 115, e.g., in accordance with a fixed or variable stimulus pattern. Moreover, the optical stimulus may be designed to provide or cause the production of nerve signals (i.e., to stimulate one or more nerves) proximate the stimulus location.

In specific non-limiting embodiments, the optical stimulation is in the form of infrared laser light that is applied transiently to a stimulus location on a patient. For example, the optical stimulation may infrared light having a wavelength of about 1800 to about 2400 nm (e.g., about 2.1 microns), and may be produced by a yttrium aluminum garnet or other suitable laser source. The minimum laser energy provided may be set to provide a desired stimulus result and may range, for example, from between about 0.3 to about 1 joule per square centimeter (J/cm²), such as from about 0.3 to about 0.6 J/cm². Alternatively or additionally, optical stimulation may be performed using other types of light sources, such as one or more light emitting diodes (LEDs), which may be pulsed in a predetermined manner, e.g., in accordance with a predetermined frequency pattern that is analogous to the haptic or other stimuli discussed above.

Of course that embodiment is but one suitable example, and other optical stimulation from other sources may also be used. For example, the optical stimulation may be in the form of light having a suitable wavelength and power to penetrate the skin of a patient, so as to stimulate one or more nerves thereof. The penetration depth of the light into and/or through a patient's skin may be a function of the wavelength of the light and the intensity (optical power) thereof, with longer wavelengths generally having larger penetration depth than shorter wavelengths.

Reference is now made to FIG. 7, which depicts one example of an optical actuator consistent with the present disclosure. As shown, optical actuator 700 may be embedded within or coupled to band 105 (e.g., to an inward facing surface 128 thereof), and may include one or a plurality of light sources 705. Light sources 705 may be configured to produce an optical stimulus in the form of specular or diffuse light, e.g., in response to control signals from controller 115. In that regard, any suitable light source may be used as light source 705. Non-limiting examples of suitable light sources include light emitting diodes (LEDs) such as laser diodes, incandescent light sources, halogen light sources, fluorescent light sources, combinations thereof, and the like. Without limitation, light sources 705 are preferably in the form of a light emitting diode and in some embodiments are or include a laser diode. Without limitation, in some embodiments at least one light source 705 is a laser diode configured to emit an optical stimulus in the form of infrared laser light, e.g., in response to control signals from control system 115.

Returning to FIG. 1, as noted above device 100 includes power source 120. In general, power source 120 functions to provide electrical power to one or more components of device 100, such as but not limited to actuators 110 and control system 115. In that regard any suitable power source may be used as power source 120. For example, in some embodiments power source 120 may be configured to couple device 100 to a source of electrical power, such as a source of AC power (e.g., mains power from a building or external power supply). Alternatively or additionally, power source 120 may be in the form of one or more batteries.

As noted above, device 100 may include a plurality of actuators 110, which may be operated continuously and/or substantial periods of time. As the operation of actuators 110 may consume significant power during their operation, power supply 120 may be configured to provide a relatively large amounts of electrical power, e.g., as compared to conventional wearable devices. Thus for example, power supply 120 may be configured or include a plurality of batteries, which may be located external to or within one or more portions of device 100. Without limitation, in some embodiments power supply 120 is an external battery or other source of power, as noted above. Alternatively, in some embodiments power supply 120 may include or be in the form of a plurality of batteries or battery cells that are housed within one or more components of device 100, such as but not limited to band 105, housing 125, control system 115, clasp 130, or a combination thereof. In the embodiment of FIG. 1, for example, power source 120 is in the form of a plurality of batteries/cells that are housed within band 105. In that regard, when band 105 includes one or more links, one or more batteries/cells of power source 120 may be included in one or more of such links.

Any suitable battery may be used as power source 120. Non-limiting examples of suitable batteries include lithium ion batteries, nickel-cadmium batteries, lead acid batteries, combinations thereof, and the like. Such batteries may or may not be rechargeable. In instances where non-rechargeable batteries are used, band 105 or another portion of device 100 may be configured to facilitate removal and replacement of such batteries. When rechargeable batteries are used, device 100 may include means to facilitate charging of power source 120, such as one or more ports for coupling power source 120 to an external source of electrical power (e.g., mains power, an external battery, etc.).

Returning again to FIG. 1, as noted above device 100 includes a control system 115, which may be housed independently or within housing 125. In such instances control system 115 may be coupled to portions of band 105, e.g., such that device 100 has a generally circumferential structure. Alternatively or additionally, control system may be provided in another part of device 100, such as within band 105, clasp 130, one or more links (not shown), or a combination thereof. In such instances it may be understood that housing 125 may be omitted, and that band 105 may define a fully or partially circumferential (e.g., circular) shape.

Regardless of where control system 115 is located, it generally functions to control the operation of device 100. In particular, control system 115 functions to control the production of stimuli by one or more of actuators 110. Accordingly, device 100 may be configured such that control system 115 can communicate with actuators 110, e.g., in a wired or wireless manner. In the former case, control system 115 may be coupled to each of actuators 110 by one or more control wires (not shown). Such control wires may extend, for example, from first connection ports proximate to the mounting location of control system 115 to second control ports proximate the location of one or more actuators 110. Alternatively or additionally, control system 115 and actuators 110 may be configured to communicate wirelessly, e.g., using one or more wireless communication protocols as noted above.

In operation, control system 115 may issue control signals to one or more actuators 110, e.g., over a wired or wireless communication link. The control signals may be configured to cause actuators 110 to produce stimuli (e.g., haptic, electrical, optical stimuli) in accordance with a fixed or variable stimulus pattern. As used herein, the term “stimulus pattern” refers to various characteristics of the stimulus to be provided to a patient wearing device 100. Non-limiting examples of such characteristics include the type of stimulus to be applied, the intensity of the stimulus, the rate (tempo) at which the stimulus is applied, the rhythm with which the stimulus is applied, the identification (e.g., location, type, etc.) of the actuator(s) to produce the stimulus, combinations thereof, and the like.

In some embodiments the control signals issued by control system 115 are configured to cause actuators 110 to produce stimuli in accordance with a stimulus pattern, i.e., so as to apply a stimulus to a stimulus location at a specified rhythm, tempo, and/or intensity. For example, the control signals in some embodiments may be configured to cause actuators 110 to produce stimuli as a pattern of pulses, wherein the pattern may have a fixed or variable rhythm, tempo, intensity, and/or location.

For example, in some embodiments the control signals may cause all of actuators 110 to produce stimulus of a fixed duration at substantially the same time, and with a substantially constant rhythm and tempo. For example, the control signals may cause all of actuators 110 to generate stimulus “pulses” at substantially the same time, wherein each pulse has a defined length, and are produced at a desired rate (e.g., a fixed number of pulses per minute).

Alternatively or additionally, the control signals may be configured such that certain actuators 110 produce stimuli at certain times, whereas other actuators 110 produce stimuli at the same or different times. For example in instances where device 100 includes first and second actuators, the c control signals issued by control system 115 may cause the first actuator to produce a first stimulus (e.g., a first pulse) at a first time period, and cause the second actuator to produce a second stimulus (e.g., a second pulse) at a second time period that is the same or different from the first time period.

In specific non-limiting embodiments, the control signals may be configured to cause actuators 110 to produce stimuli in accordance with a stimulus pattern that is based on a melody and/or music, such as a song. For example, control system 115 may produce control signals that cause actuators 110 to produce stimuli in accordance with the “beat” of a song, e.g., with or without syncopation of the beat. While such stimulus patterns may be generated manually, in some embodiments control system may be configured to generate them automatically, e.g., from a source of audio information. By way of example, a patient or medical professional may upload music data file (e.g., MPEG Layer III Audio files, Windows Media Audio files, Free Lossless Audio Codec files, and the like) to a memory of control system 115, after which control system 115 may process the file to produce a stimulus pattern corresponding thereto.

As may be appreciated, the characteristics (tempo, intensity, rhythm, etc.) of the stimulus produced by the first and second actuators may be individually controlled by controller 115. Thus, controller 115 may control device 100 such that actuators 110 are active at the same or different times, produce different types of stimuli, produce stimuli at different rates or with a different tempo, etc. Likewise, controller 115 may produce control signals that cause actuators 110 to produce stimuli in a specified order, e.g., such that the stimuli are produced at one location or a plurality of locations around band 105. In some instances, control signals from controller 115 may be configured to cause a plurality of actuators 115 to actuate in sequence (i.e., one after the other) such that a produce stimulus “moves around” band 105 in accordance with a desired stimulus pattern. In any case, the stimulation pattern implemented by control system 115 may result in control signals that are configured to cause actuators 110 to produce stimuli as one or a plurality of pulses.

With the foregoing in mind, control system 115 may be in the form of or include a programmable or non-programmable circuit that is configure to generate control signals in accordance with at least one predefined stimulus pattern. For example, control system may include one or a plurality of hardware circuits, wherein each hardware circuit is configured to produce control signals in accordance with one or more fixed stimulus patterns. In some instances to provide variability, control system 115 may include multiple hardware circuits, wherein each hardware circuit is configured to produce control signals in accordance with a single predefined stimulus pattern that differs from the predefined stimulus patterns that produced by other hardware circuits.

To introduce further variability, control system 115 may include a user interface which may allow a patient or speech therapist to change various characteristics of a stimulus pattern to be applied. For example, the user interface may enable a patient/speech therapist to alter the intensity, tempo, etc. of the stimulus pattern, as desired. In some embodiments, the user interface includes components such as variable resistors, variable capacitors, and the like, which may be altered by a patient or other user, e.g., to adjust characteristics of a stimulus pattern. In such instances the user interface may include, for example, tactile inputs such as push buttons, slide switches, rotating switches, combinations thereof, and the like. Though such inputs, a patient or other user may select a stimulation pattern for use, and may alter various characteristics such as those noted above as desired. Of course, other types of inputs (e.g., a touch screen, soft buttons, combinations thereof, and the like may also be used.

Alternatively or additionally, control system 115 may be programmable. In such embodiments control system 115 may be programmed or otherwise configured to produce control signals in accordance with one or more predefined stimulus patterns, or in accordance with a custom stimulus pattern specified by a patient, speech therapist, or other user. Programming of control system 115 may be accomplished via a user interface, e.g., tactile inputs, soft inputs, a touch screen, etc. as described above. Though the user interface, a patient other use may select, augment, create, or change a stimulus pattern as desired.

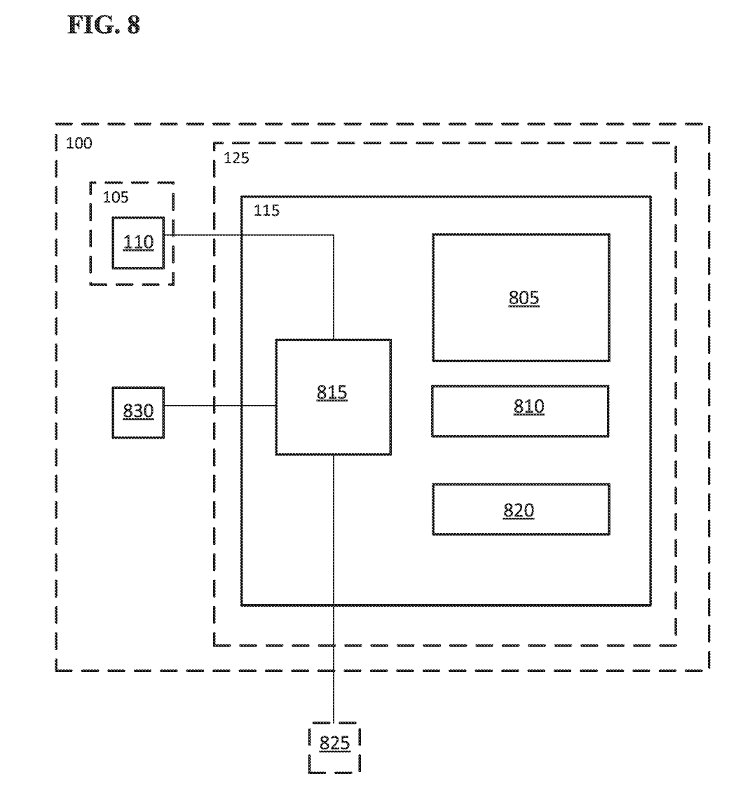

Reference is therefore made to FIG. 8, which depicts one example of a control system 115 consistent with the present disclosure. As shown, control system 115 may be disposed within or otherwise coupled to optional housing 125 and/or to device 100. In any case, control system 115 includes a processor 805, a memory 810, and communications (COMMS) interface 815.

Processor 805 may be any suitable general purpose processor or application specific integrated circuit, and may be capable of executing one or multiple threads on one or multiple processor cores. Without limitation, in some embodiments, processor 305 is a general purpose processor, such as but not limited to the general purpose processors commercially available from INTEL® Corp., ADVANCED MICRO DEVICES®, ARM®, NVIDIA®, APPLE®, SAMSUNG®, and TEXAS INSTRUMENTS®. In other embodiments, processor 805 may be in the form of a very long instruction word (VLIW) and/or a single instruction multiple data (SIMD) processor (e.g., one or more image video processors, etc.).

Memory 810 may be any suitable type of computer readable memory (including, but not limited to, non-transitory computer readable memory). Example memory types that may be used as memory 810 include but are not limited to: semiconductor firmware memory, programmable memory, non-volatile memory, read only memory, electrically programmable memory, random access memory, flash memory (which may include, for example NAND or NOR type memory structures), magnetic disk memory, optical disk memory, combinations thereof, and the like. Additionally or alternatively, memory 810 may include other and/or later-developed types of computer-readable memory. Without limitation, in some embodiments, memory 810 is configured to store data such as computer readable instructions in a non-volatile manner.

Processor 805 is generally configured to communicate with memory 810 and to execute instructions stored thereon. Based on those instructions, processor 805 may cause control signals to be transmitted to one or more actuators 110, e.g., disposed within or on band 105. As noted above, the control signals may be communicated to actuators 110 over a wired or wireless communication link, e.g., in accordance with one or more known or future developed wireless communications protocols. The control signals may be configured to cause actuators 110 to produce stimuli in accordance with one or more stimulus patterns, as described above.

In various embodiments, the memory 810 may include computer readable instructions that when executed by processor 805 cause control system 115 to generate control signals that regulate the characteristics of the stimuli produced by one or more actuators 110. For example, the instructions may cause control system 115 to issue control signals that specify the rhythm, tempo, and/or intensity of stimuli generated by actuators 110. Moreover, the instructions may cause control system to configure such control signals such that actuators 110 produce stimuli in a specified order. For example, the control signals may be configured to cause actuators 110 to produce stimuli in the form of a pulse pattern that emulates or is based at least in part on a song, a melody, a stimulus pattern defined by a patient, speech therapist, or another user, combinations thereof, and the like.

For example, the instructions when executed may cause control system 115 to configure control signals to cause actuators 110 to produce stimuli in accordance with first and second stimulus patterns, wherein the first and second stimulus patterns differ in some way. For example, the first stimulus pattern may be a training pattern with characteristics that are suitable for training an untrained patient to use device 100, whereas the second stimulus pattern may be a maintenance pattern that may be suitable for use by trained patients, e.g., in an out of the doctor's office setting. Depending on the patient, the training pattern and maintenance pattern may differ in various ways, such as in the tempo of stimulation, intensity of stimulation, type of stimulation, location of stimulation (i.e., stimulus location on the patient's body), combinations thereof, and the like.

COMMS 815 is generally configured to enable control system 115 to transmit and receive data. For example, COMMS 815 may be configured to receive stimulus patterns and other data from an external device 825, e.g., via a wired or wireless communications link. External device 825 may be any suitable device, such as an external server, desktop computer, laptop computer, mobile phone, smart phone, tablet computer, combination thereof, and the like. Likewise, COMMS 815 may be configured to enable control system 115 to transmit control signals to one or more actuators 110, in accordance with a wired or wireless communication protocol. COMMS 815 may therefore include hardware to support wired and/or wireless communication, e.g., one or more transponders, antennas, BLUETOOTH™ chips, personal area network chips, near field communication chips, wired and/or wireless network interface circuitry, combinations thereof, and the like. In some embodiments, COMMS 815 may allow speech therapy device 100 to be substantially controlled by external device 825 for example, by use of an application running on external device 825.

Device 100 may also include one or more sensors that may be leveraged to monitor various characteristics of a patient wearing device 100, or the efficacy of stimulus patterns produced by actuators 110 in response to control signals issued by control system 115. This concept is shown in FIG. 8, which depicts device 100 as including one or more sensors 830. It is noted that while FIG. 8 depicts an embodiment in which sensor(s) 830 is/are disposed within device 100 and external to control system 115, that configuration is not required. Indeed in some instances, one or more sensors 830 may be disposed on or within control system 115, on or within housing 125 (but external to control system 115), on or within band 105, on or within clasp 130, or at some other suitable locations.

With the foregoing in mind, a wide variety of sensors may be used as sensors 830. Non-limiting examples of suitable sensors that may be used include acoustic sensors (e.g., a microphone),

Control system 115 may also be configured to monitor at least one contextual information signal and to evaluate the effectiveness of an applied stimulus pattern on the speech of a patient wearing device 100. The contextual information may include, for example, information from one or more sensors that are disposed internal or external to device 100. In that regard and as shown in FIG. 8, in some embodiments device 100 may include one or more sensors 830, each of which may be configured to provide contextual information in one or more contextual information signals to control system 115. Of course, sensors 830 need not be within device 100, and may be disposed at any suitable location external to device 100.

Based on contextual information provided by sensors 830, control system 115 may dynamically adjust one or more aspects of a stimulus pattern, e.g., by changing the stimuli produced by actuators 110 in some manner. For example, based on contextual information provided by one or more sensors 830, controller 115 may adjust the rate, tempo, intensity, pattern, or other characteristic of an applied stimulus pattern, or may switch from a first stimulus pattern to a second, different stimulus pattern. Alternatively, control system 115 may generate a new stimulus pattern based on received contextual information. More generally, control system 115 may be configured to monitor and to alter or replace an applied stimulus pattern based at least in part on contextual information provided by one or more sensors. For example when control system 115 detects the occurrence of a triggering event (e.g., excessive delay in speech initiation, stuttering, etc.), it may alter or replace an applied stimulus pattern to compensate accordingly.

Control over an applied stimulus pattern may be performed with a stimulus control module (SCM) 820, which may be included in control system 115 as shown in FIG. 8. SCM 820 may, for example, be in the form of hardware or logic implemented at least in part in hardware to perform stimulus control operations consistent with the present disclosure. Alternatively or additionally, SCM 820 may include or be in the form of a computer readable storage medium (in which case, e.g., SCM 820 may be maintained in memory 810) including instructions which when executed by a processor (e.g., processor 805) of device 100, cause device 100 to perform stimulus control operations consistent with the present disclosure.

Sensor(s) 830 may be any suitable sensor for detecting and/or taking measurements of contextual information which may be correlated to the effect of a stimulus pattern applied by actuators 110 on one or more aspects of a patient's speech. Non-limiting examples of such contextual information includes acoustic information such as but not limited to recordings of a patient's speech, user activity information (e.g., biometric information such as a user's heart rate, blood pressure, blood oxygen level, presence or absence of sweat, body temperature, etc.), nerve/muscle actuation information such as electromyography data, brain activity information such as electroencephalography data, combinations thereof, and the like. Sensor(s) 830 may therefore be in the form of an acoustic sensor (e.g., a microphone), a biosensor, or a combination thereof. Non-limiting examples of biosensors that may be used in sensor(s) 803 include a heart rate sensor, a blood pressure sensor, a blood oxygen level sensor (e.g., a pulse oximetry sensor), a body temperature sensor, a sweat sensor, a electromyography sensor, an electroencephalography sensor, combinations thereof, and the like. Without limitation, in some embodiments sensor(s) 830 include at least one acoustic sensor, such as a microphone.

It is noted that while sensor(s) 830 is/are shown in FIG. 8 as integrated with device 100, such a configuration is not required. Indeed, the present disclosure envisions embodiments in which sensor(s) 830 is/are not integrated with device 100, except insofar as it/they may be in wired or wireless communication with device 100. For example in some embodiments, device 100 may in the form of a wearable device (e.g., a wrist worn wearable) that is in wired or wireless communication with an external computing device, wherein one or more of sensor(s) 830 are disposed in or are coupled to the external computing device.

Sensor(s) 830 may be configured to detect or otherwise obtain contextual information and transmit one or more contextual information signals to SCM 820. In general, the contextual information signals may be configured to cause SCM 820 to determine an adjusted stimulus pattern based at least in part on the contextual information detected or otherwise reported by sensor(s) 830.

In some embodiments the contextual information detected by sensor(s) 830 may correspond to and/or otherwise be one or more aspects of a patient's speech. In embodiments in which sensor(s) 830 include an acoustic sensor such as a microphone, data produced by the acoustic sensor may include information regarding the speech of a patient wearing device 100. For example, data produced by the acoustic sensor may include a recording of a patient's speech. In such instances, SCM 820 may analyze received acoustic information from sensor(s) 830 to determine the effectiveness of an applied stimulus pattern. For example, SCM 820 may analyze the reported acoustic data to determine the cadence of a user's speech, frequency of word initiation, or other characteristics which may be indicative of the effectiveness of an applied stimulus pattern. SCM 820 may accomplish this, for example, by comparing recorded audio information from sensor(s) 830 against a reference, such as an (e.g., calibrated) audio recording, of normal (or normalized) human speech, previous recordings of a patient's speech, combinations thereof, and the like. As may be appreciated, the reference audio recording may be supplied or configured by a health care professional, such as the patient's doctor, speech therapist, or the like.

In some embodiments, sensors 830 include at least an acoustic sensor (e.g., microphone) that is configured to record the speech of a patient wearing device 100. In such instances the data recorded by the acoustic sensor 330 may be used for training purposes. For example, speech therapy device 100 and/or external device 325 may include a training mode. During a training exercise, a patient may be prompted by speech therapy device 100 and/or external device 325 to speak predetermined words and/or phrases. The acoustic sensor may detect the words spoken by the patient and produce corresponding contextual information (e.g., a recorded audio signal) that is saved to memory 310. Speech therapy device 100 may then compare the speech pattern to a previously recorded speech pattern or a baseline speech pattern (e.g., a speech pattern recorded by a speech therapist). Based on this comparison, speech therapy device 100 may provide feedback to a patient and/or a medical professional. In some embodiments, when in the training mode, speech therapy device 100 and/or external device 326 may prompt the user to speak random words and/or phrases instead of predetermined words and/or phrases. The data gathered during the training mode may be used to generate new and/or adjust preexisting pulse patterns.

Based on such analysis, SCM 820 may determine whether a patient's speech is within or outside a desired tolerance, relative to the reference audio recording. If the patient's speech is within tolerance, SCM 820 may cause controller 115 to continue to implement a current stimulus pattern. If the patient's speech is outside a desired tolerance, however, SCM 820 may cause controller 115 to vary one or more aspects of a current stimulus pattern and monitor contextual information provided by sensor(s) 830 to determine the effect of the change. The manner in which the stimulus pattern may be changed may also be predetermined, for example, by a health care professional. The contextual data recorded by sensors 830 and the analysis and adjustments made by controller 115 may be stored, for example, in memory 810, and may be later retrieved (e.g., by a health care professional or other individual) for review.

In some embodiments, control system 115 may also monitor the data from one or more sensors 830 (e.g., an acoustic sensor) for a disrupted speech pattern. A disrupted speech pattern may generally be considered any disruption in the cadence and/or natural flow of the speech of the patient, e.g., relative to a normalized or reference speech pattern. A disrupted speech pattern may, for example, include a long pause between spoken words and/or stuttering. In response to the detection of a disrupted speech pattern control system 115 may place a disrupted speech pattern indicator in contextual data recorded in memory 810 (e.g., in recorded audio data). The disrupted speech pattern indicator may allow the user and/or therapist to quickly determine the effectiveness (or lack thereof) of an applied stimulus pattern on a patient's speech. Alternatively, or additionally, device 100 may be configured to allow a patient or other user to manually input disrupted speech pattern indicators by, for example, pressing a button (digital or physical) or activating a sensor on speech therapy device 100 when the disrupted speech pattern is occurring.

In that way, controller 115 may dynamically adjust an applied stimulus pattern as device 100 is worn by a user, analyze the impact of the applied adjustments, and store such information in a memory of controller 115 for later review. As may be appreciated, such capability may provide numerous advantages relative to traditional in-office speech therapy sessions. For example, such capability may allow for a patient to undergo speech therapy training without the need for a speech therapist or other health care professional to be present. For example, a speech therapist or other health care professional may configure device 100 for use at home by the patient. As the patient uses device 100 it may determine the impact of an applied stimulus pattern (e.g., from contextual information), modify an applied stimulus pattern, determine the results of the modification, and store the contextual data, adjustments, etc. in memory for later review by the patient and/or health care professional.

It is noted that for the sake of clarity the above discussion focuses on instances in which SCM 830 adjusts one or more aspects of an applied stimulus pattern (e.g., haptic, electrical, optical stimulus intensity, stimulus rate, stimulus tempo, stimulus location, etc.) based on a single type of contextual information, namely acoustic information such as a recording of a patient's speech. It should be understood that such discussion is for the sake of example, and that the operations of SCM 830 are not limited to such implementations. Indeed the present disclosure envisions embodiments wherein SCM 830 leverages a combination of different types of contextual information to determine appropriate adjustments to an applied stimulus pattern.

For example, in some embodiments sensor(s) 830 may include sensors that provide contextual information relevant to a patient's activity level. Such sensors may include, for example, an accelerometer, a gyroscope, or a combination thereof. In such embodiments SCM 830 may, in response to receipt of a contextual information signal containing accelerometer data and gyroscope data, calculate or otherwise determine an adjusted stimulus pattern. For example, in response to such contextual information, SCM 830 may dynamically adjust the intensity of the stimuli produced by actuators 110 to account for the impact of user activity on the ability of the user to detect/react to the applied stimulus. For example, where contextual information suggests a high level of user activity (e.g., the patient is walking, running, etc.), SCM 830 may increase the intensity of stimuli produced by actuators 830 to account for a possible reduction in sensitivity of the patient to lower intensity stimuli. Additionally or alternatively, control system 115 may adjust characteristics of an applied stimulus pattern (e.g., the rate, intensity, and/or order) in response to a user input, e.g., received through a user interface of device 100.

While the above discussion focuses on embodiments in which an SCM local to device 100 receives and analyzes contextual data to determine adjustments to an applied stimulus pattern, it should be understood such a configuration may not be required. For example, in some embodiments contextual information from a sensor 830 may be communicated (e.g., by control system 115 or another component) to external device 825 for analysis. In such embodiments, external device 825 may include an SCM which functions in substantially the same manner as SCM 820 above. That is, an SCM in external device 825 may process contextual information (e.g., speech patterns, audio recordings, biosensor data, etc.) to determine whether an adjustment to an applied stimulus pattern is desired. If so, the SCM may cause the external device 825 to transmit an updated stimulus pattern configuration to control system 115 for implementation.

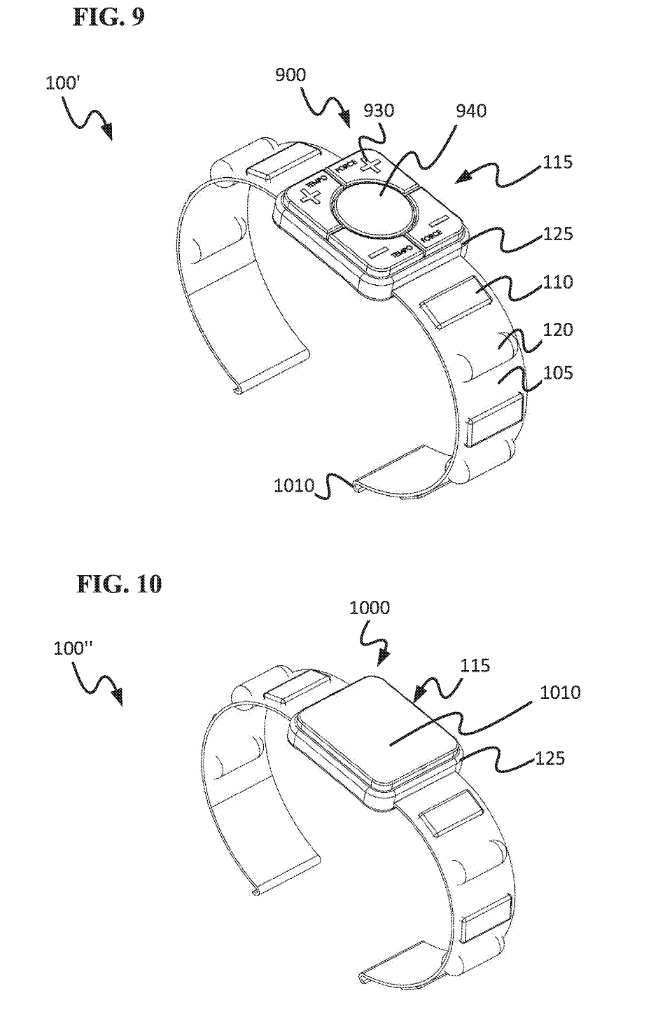

Reference is now made to FIGS. 9 and 10, which depict non-limiting example embodiments of a speech therapy device consistent with the present disclosure. More specifically, FIGS. 9 and 10 illustrate two speech therapy devices which operate in substantially the same manner as described above, but which differ in the manner by which a patient or other user may interact with the functions of control system 115. Specifically, FIG. 10 depicts a speech therapy device 100′ that includes a band 105, actuators 110, control system 115, power source 120, and housing 125, the nature and function of which have been previously described. As further shown, device 100′ includes a user interface 900 that includes physical buttons to enable a patient or other user to interact with the functions of control system 115. For example, user interface 900 includes tactile inputs (e.g. buttons) 930 that are configured to enable a patient or other user to adjust elements of a stimulus pattern to be applied by device 100′, e.g., the rate, intensity, etc. of stimuli produced by actuators 110. In addition, user interface 900 includes a power input (e.g., button) 940, which is configured to enable a patient or other user to turn device 100′ ON or OFF.

Of course the devices described herein need not employ physical buttons to facilitate interaction with the functions of control system 115. For example, in some embodiments the devices described herein include a touch screen or other soft-button interface that is configured to facilitate such interaction. This concept is generally shown in FIG. 10, which depicts a speech therapy device 100″ as including a user interface 1000 that includes a display 1010, such as a touch screen. As may be appreciated, various software elements corresponding to functions of control system 115 may be produced on touch screen 1010, thereby enabling a patient or other user to configure the operation of device 100″.

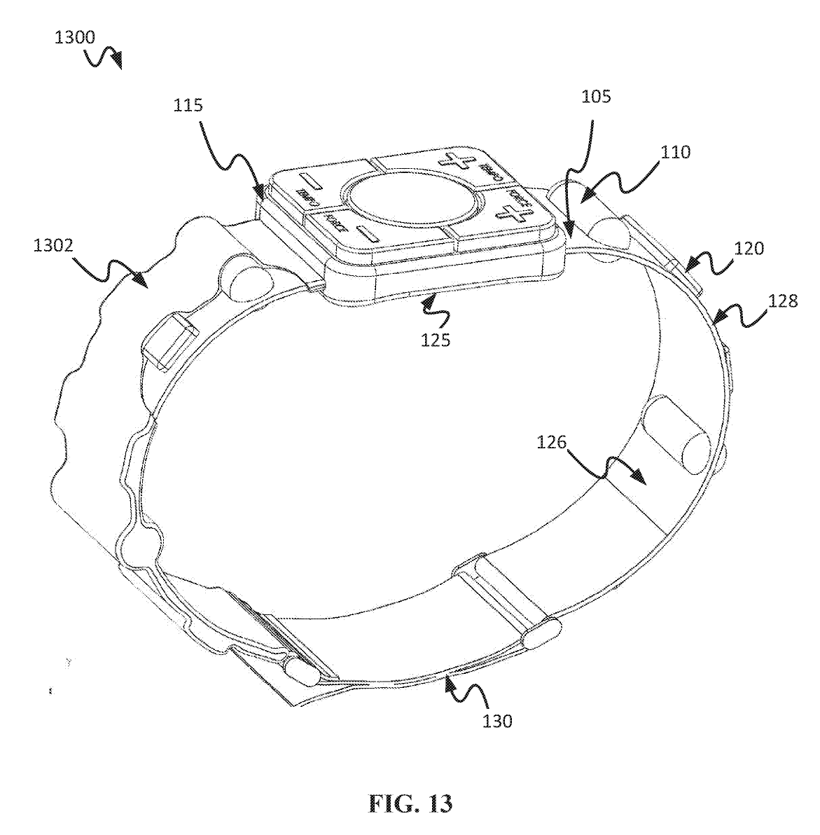

It is noted that while the foregoing discussion focuses on embodiments in which actuators and/or various other components of device 100 may be in direct contact with the skin of a user, such a configuration is not required. Indeed, the present disclosure envisions embodiments in which various components of band 105, control system 115, and/or housing 125 are insulated from the skin of a wearer, e.g., by a covering material. This concept is illustrated in FIG. 13, which depicts another example of a speech therapy device 1300 consistent with the present disclosure. As shown in FIG. 13, speech therapy device 1300 includes many of the same elements as speech therapy devices 100 and 1200, and therefore the nature and operation of such components is not described again in the interest of brevity. As further shown, speech therapy device 1300 also includes cover 1302, which in this case covers a variety of components within band 105, such as actuators 110 and power sources 120.

Cover 1302 may be made of any suitable material, such as but not limited to rubber, elastomeric polymers, thermoplastic polymers, thermosetting polymers, carbon fiber, metal, or any other suitable material. Without limitation, in come embodiments cover 1302 is formed from or includes a non-conductive material, such as a rubber or non-conductive elastomeric or polymer material, such as but not limited to a rubber, a polyurethane, or the like.

Although not shown in FIG. 13, cover 1302 may be configured to facilitate transmission of stimuli produced by actuators 110 to a patient. By way of example, cover 1302 may include openings, regions of different hardness, surface features, or the like, which facilitate transmission of haptic, electrical, or optical stimuli to a wearer of device 1300.

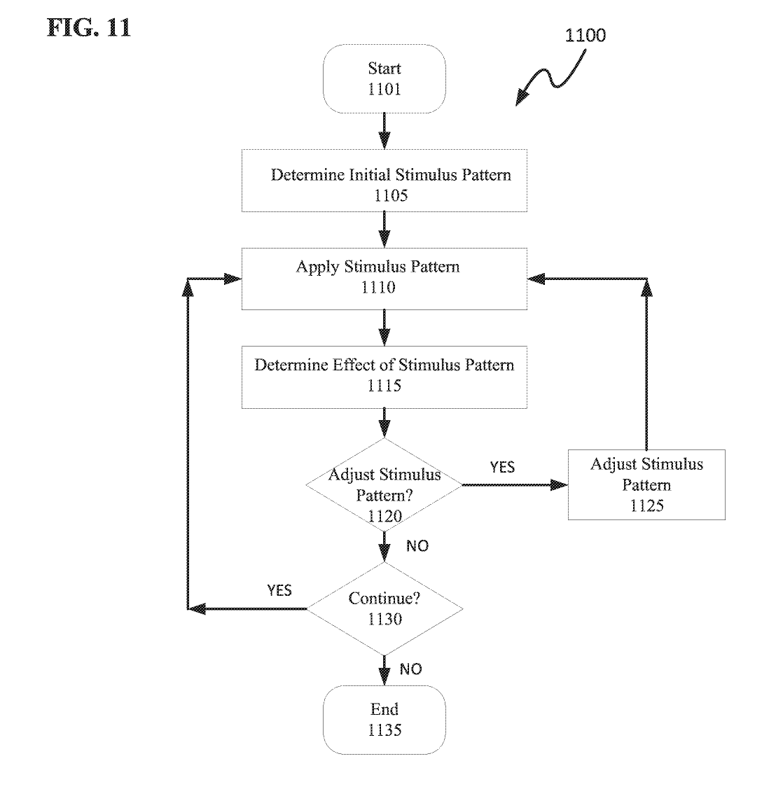

Another aspect of the present disclosure relates to speech therapy methods using a speech therapy device such as those described above. In that regard reference is made to FIG. 11, which is a flow chart of example operations in accordance with a speech therapy method consistent with the present disclosure.

As shown, the method 1100 begins at block 1101. The method may then proceed to block 1105, pursuant to which an initial stimulus pattern may be determined. As discussed previously, the stimulus pattern may include a defined rate, intensity, and/or order of stimulus generation, e.g., by one or more actuators of a speech therapy device consistent with the present disclosure. Once an initial stimulus pattern has been determined (or previously determined) the method may proceed to block 1110. Pursuant to block 1110, the stimulus pattern may be applied to a patient, e.g., with a device consistent with the present disclosure. For example, a control system of such a device may produce control signals to cause the production of stimuli in accordance with the stimulus pattern, e.g., by one or more actuators.

During or after the application of the stimulus pattern, the method may proceed to block 1115, pursuant to which the effect of the applied stimulus pattern may be determined. For example and as described above, during application of a stimulus pattern one or more sensors of a speech therapy device may record contextual data, such as a recording of a patient's speech. The contextual data may then be provided to a control system of the device (e.g., an SCM thereof) or an external device, which may analyze the contextual data to determine the effectiveness of the applied stimulus pattern. For example, the control system and/or external device may analyze the contextual information to determine the occurrence of a triggering event, such as a disputed speech pattern, threshold deviation from a reference speech pattern, biological information, etc.

The method may then proceed to block 1120, pursuant to which a decision may be made as to whether an adjustment to the stimulus pattern is desired. As noted previously, such adjustments may include altering one or more characteristics of an applied stimulus pattern (e.g., intensity, rate, etc.), replacing an applied stimulus pattern with a different stimulus pattern, combinations thereof, and the like. As explained above, the outcome of determination 1120 may be conditioned on the detection of a triggering event or some other criterion.

If an adjustment to an applied stimulus pattern is to be made, the method may proceed from block 1120 to block 1125, pursuant to which the relevant adjustment may be implemented, e.g., by a control system of a speech therapy device consistent with the present disclosure. The method may then loop back to block 1110, pursuant to which the adjusted stimulus pattern may be applied.

If an adjustment to an applied stimulus pattern is not to be made, however, the method may proceed from block 1120 to block 1130. Pursuant to block 1130, a decision may be made as to whether the method is to continue. If so, the method may loop back to block 1110. But if not, the method may proceed from block 1130 to block 1135 and end.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

What is claimed is:

1. A neurologic therapy device comprising:
a band;
at least one actuator coupled to the band;
a power source; and
a control system;
wherein:
said control system is configured to generate a control signal for said at least one actuator in accordance with a stimulus pattern;
in response to said control signal, said at least one actuator is configured to produce one or more stimuli in accordance with said stimulus pattern;
when said band is worn by a patient, said at least one actuator is configured to apply said stimuli to a stimulus location on a body of said patient, said stimuli being configured to stimulate one or more nerves proximate said stimulus location; and
said stimulus pattern is configured to assist the patient to initiate and cadence their speech.

2. The neurologic therapy device of claim 1, further comprising a housing coupled to said band, wherein said control system is disposed within said housing.

3. The neurologic therapy device of claim 1, wherein:
said band comprises an inward facing surface and an outward facing surface, wherein the inward facing surface is proximate to a body part of the patient when said device is worn; and
said at least one actuator is disposed on said inward facing surface.

4. The neurologic therapy device of claim 1, wherein when said device is worn on a wrist of a patient, said at least one actuator is disposed on a volar, doral, or lateral side of said wrist.

5. The neurologic therapy device of claim 1, wherein said stimulus pattern is a pre-determined pattern that is based at least in part on a melody of a song.

6. The neurologic therapy device of claim 1, wherein:
said at least one actuator comprises a plurality of actuators;
when said band is worn on said body part of said patient, said plurality of actuators are disposed around said body part; and
said control signals cause said plurality actuators to produce a stimulus in a sequence that moves around the body part of said patient.

7. The neurologic therapy device of claim 1, wherein said at least one actuator is selected from a haptic actuator, electrical actuator, optical actuator, and a combination of two or more thereof.

8. The neurologic therapy device of claim 7, wherein said at least one actuator is at least one haptic actuator.

9. The neurologic therapy device of claim 1, wherein said control system is configured to generate said control signal independent said patient speaking.

10. The neurologic therapy device of claim 9, further comprising at least one sensor for providing contextual feedback to said control system, wherein said control system is configured to provide generate said control signal independent of said contextual feedback.

11. A neurologic therapy method, comprising:
causing a patient to wear a therapeutic device, the neurologic therapy device comprising:
a band;
at least one actuator coupled to the band;
a power source; and
a control system;
identifying a stimulus pattern to be implemented by said control system;
generating, with said control system, a control signal in accordance with said stimulus pattern;
transmitting said control signal to said at least one actuator, wherein said control signals are configured to cause said at least one actuator to produce stimuli in accordance with said stimulus pattern; and
stimulating at least one nerve in a body part of the patient with said stimuli;
wherein said stimulus pattern is configured to assist the patient to initiate and cadence their speech.

12. The neurologic therapy method of claim 11, wherein said neurologic therapy device further comprises a housing coupled to the band, and the control system is disposed within the housing.

13. The neurologic therapy method of claim 11, wherein:
said band comprises an inward facing surface and an outward facing surface, wherein the inward facing surface is proximate to a body part of the patient when said device is worn; and
at least a portion of said at least one actuator is disposed on said inward facing surface.

14. The neurologic therapy method of claim 11, wherein causing said device to be worn by a patient comprises causing the patient to wear the device about a wrist, such that said at least one actuator is disposed on volar, doral, or lateral sides of said wrist.

15. The neurologic therapy method of claim 11, wherein said stimulus pattern is is a pre-determined stimulus pattern and is based at least in part on a melody of a song.

16. The neurologic therapy method of claim 11, wherein:
said at least one actuator comprises a plurality of actuators;
causing said device to be worn by a patent comprises causing the patient to wear the device about a body part, such that said plurality of actuators are disposed around said body part; and
said control signals are configured to cause said plurality actuators to produce a stimulus in a sequence that moves around the body part of said patient.

17. The neurologic therapy method of claim 11, wherein said at least one actuator is selected from a haptic actuator, electrical actuator, optical actuator, and a combination of two or more thereof.

18. The neurologic therapy method of claim 17, wherein said at least one actuator includes at least one a haptic actuator.

19. The neurologic therapy method of claim 11, wherein said control signals are generated independent of said patient speaking.

20. The neurologic therapy method of claim 19, further comprising receiving contextual information from a sensor in communication with the control system, wherein said control signals are generated independently of said contextual information.
